(19) United States
(12) Patent Application Publication
(10) Pub. No.: US 2012/0288442 A1
(43) Pub. Date: Nov. 15, 2012

(54) Nuclear Imaging Method Using Molecular Target Detection Agent for Liver Fibrosis

(75) Inventors: Chun-Chia Cheng, Taipei County (TW); Shui-Cheng Lee, Taoyuan County (TW); Pei-Chih Ku, Taoyuan County (TW)

(73) Assignee: ATOMIC ENERGY COUNCIL-INSTITUTE OF NUCLEAR ENERGY RESEARCH, Taoyuan County (TW)

(21) Appl. No.: 13/105,019

(22) Filed: May 11, 2011

(51) Int. Cl. A61K 51/10; A61K 49/00

(52) U.S. Cl. 424/1.69; 424/9.6

(57) ABSTRACT

A method for diagnosing liver fibrosis is provided. A monoclonal antibody and an antagonist of cannabinoid receptor in liver fibrosis cell are labeled. The monoclonal antibody or the antagonist is injected for nuclear imaging. Thus, through the image obtained through the nuclear imaging, liver fibrosis is diagnosed and traced in clinic use for preventive medicine.

### TECHNICAL FIELD OF THE DISCLOSURE

The present disclosure relates to nuclear imaging; more particularly, relates to providing a safe, non-invasive and accurate diagnosis and trace for liver fibrosis to achieve early diagnosis and early treatment for preventive medicine.

### DESCRIPTION OF THE RELATED ARTS

Until now, liver fibrosis is mainly diagnosed with tissues obtained through liver puncture for dyeing to be examined by the naked eyes of a pathologist. This method is dangerous in a certain degree. In addition, it is a very subjective examination and sampling error in liver puncture may be very big. Hence, accuracy of the examination is not high.

In 2006, Xundi Xu, et. el suggested that cannabinoid receptor 1 (CB1) can be used as a prognosis factor for diagnosing hepatocellular carcinoma (HCC). Yet, its mechanism is not clear. Hence, the prior arts do not fulfill all users' requests on actual use.

### SUMMARY OF THE DISCLOSURE

The main purpose of the present disclosure is to provide a safe, non-invasive and accurate liver fibrosis diagnosis and trace in clinic use to achieve early diagnosis and early treatment for preventive medicine.

The second purpose of the present disclosure is to provide a marker of monoclonal antibody of CB1 labeled with a radioactive nuclide like Tc-99m, I-123, I-125, In-111 or Re-188 for nuclear imaging for liver fibrosis.

The third purpose of the present disclosure is to provide a marker of antagonist of CB1 labeled with a fluorescent substance for nuclear imaging for liver fibrosis.

The fourth purpose of the present disclosure is to obtain locations and distributions of liver fibrosis and further a liver fibrosis level through brightness of image obtained through nuclear imaging.

To achieve the above purposes, the present disclosure is a nuclear imaging method using a molecular target detection agent for liver fibrosis, comprising steps of: (a) obtaining a monoclonal antibody or an antagonist of cannabinoid receptor of liver fibrosis cell; (b) obtaining a marker through labeling the monoclonal antibody or the antagonist with a radioactive isotope or a fluorescent substance, respectively; and (c) putting the marker into a body through intravenous injection and processing an in vivo imaging to the body. Accordingly, a novel nuclear imaging method using a molecular target detection agent for liver fibrosis is obtained.

### BRIEF DESCRIPTIONS OF THE DRAWINGS

The present disclosure will be better understood from the following detailed description of the preferred embodiment according to the present disclosure, taken in conjunction with the accompanying drawings, in which

FIG. 1 is the flow view showing the preferred embodiment according to the present disclosure;

FIG. 2A is the view showing the microSPECT/CT image of the mouse having liver fibrosis;

FIG. 2B is the view showing the microSPECT/CT image of the normal mouse;

FIG. 2C is the view showing the biodefense results of the normal mouse and the mouse having liver fibrosis;

FIG. 3A is the view showing the structure of Tocrifluor;

FIG. 3B is the view showing the image of the mouse having liver fibrosis taken through fluorescence imaging; and

FIG. 3C is the view showing the image of the normal mouse taken through fluorescence imaging.

### DESCRIPTION OF THE PREFERRED EMBODIMENT

The following description of the preferred embodiment is provided to understand the features and the structures of the present disclosure.

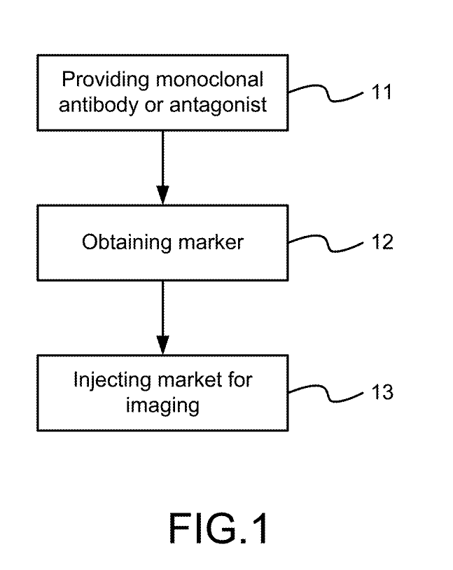

Please refer to FIG. 1, which is a flow view showing a preferred embodiment according to the present disclosure. As shown in the figure, the present disclosure is a nuclear imaging method using a molecular target detection agent for liver fibrosis, where liver fibrosis is diagnosed by identifying cannabinoid receptor 1 (CB1) contained inside through immunohistochemistry (IHC) or fluorescence imaging, comprising the following steps:

(a) Providing monoclonal antibody or antagonist 11: A monoclonal antibody or an antagonist of cannabinoid receptor in liver fibrosis cells is provided.

(b) Obtaining marker 12: A marker is obtained through labeling the monoclonal antibody or the antagonist with a radioactive isotope or a fluorescent substance, respectively.

(c) Injecting market for imaging 13: The marker of the monoclonal antibody labeled with the radioactive isotope or that of the antagonist labeled with the fluorescent substance is put into a human's or animal's body through intravenous injection. Then, an in vivo imaging of the body is processed for biodefense (BIOD) identification. Therein, the in vivo imaging is done through computed tomography (CT) or fluorescence imaging, where the CT can be positron emission tomography (PET) or single photon emission computed tomography (SPECT).

Thus, a novel nuclear imaging method using a molecular target detection agent for liver fibrosis is obtained.

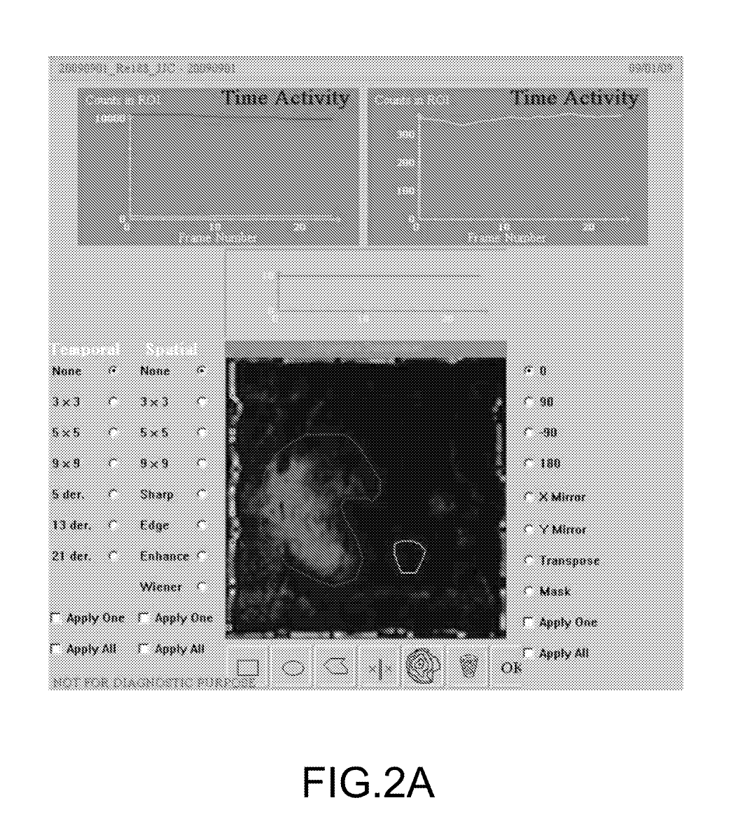

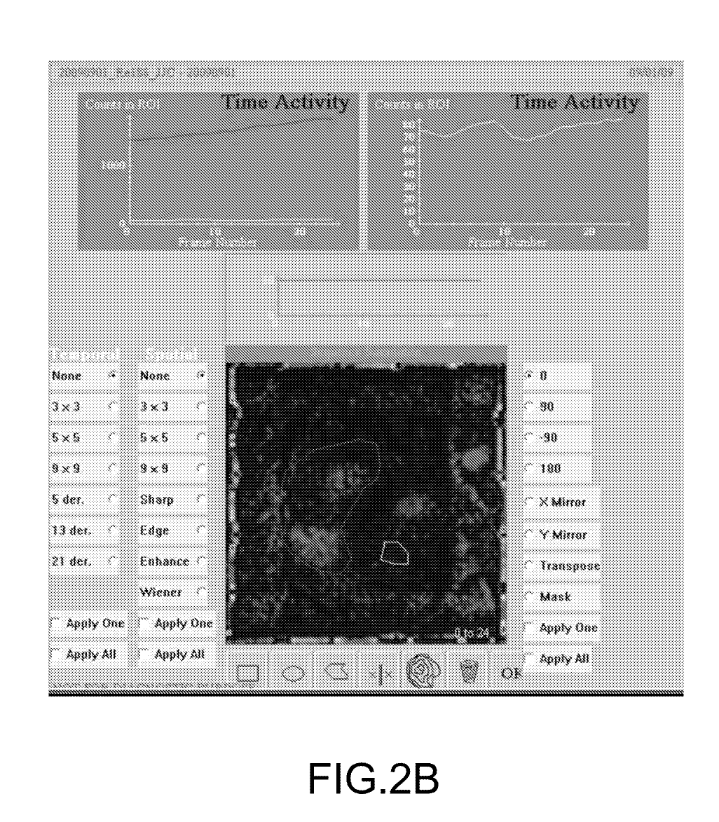

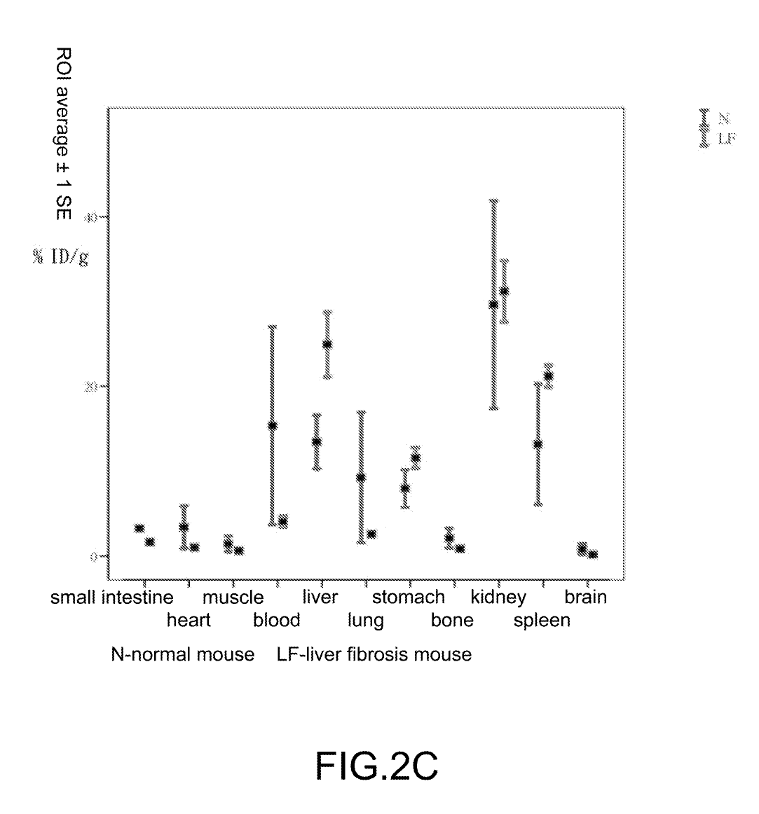

Please refer to FIG. 2A to FIG. 2C, which are views showing microSPECT/CT images of a mouse having liver fibrosis and a normal mouse; and a view showing biodefense results of the normal mouse and the mouse having liver fibrosis. As shown in the figures, a radioactive isotope used for labeling a monoclonal antibody of a liver fibrosis cell to obtain a marker can be a radioactive nuclide of Tc-99m, I-125, I-123, In-111 or Re-188. A normal mouse and a mouse having liver fibrosis are injected with 300 uL 2 mg/mL CB1 Cannabinoid Receptor Antibody-600 uCi Re-188 for imaging for 30 minutes (min) after 30 min to identify CB1 in liver fibrosis cells through IHC. As results show in FIG. 2A and FIG. 2B, regions of Interest (ROI) have a number of 10000 in the mouse having liver fibrosis and a number of only 1300 in the normal mouse, where a ROI ratio between the two mice is 7.69. After the imaging, BIOD identification is processed as results shown in FIG. 2C, where differences between the mouse having liver fibrosis and the normal mouse are shown and locations and distributions of liver fibrosis can be effectively detected with the coordination of microSPECT/CT.

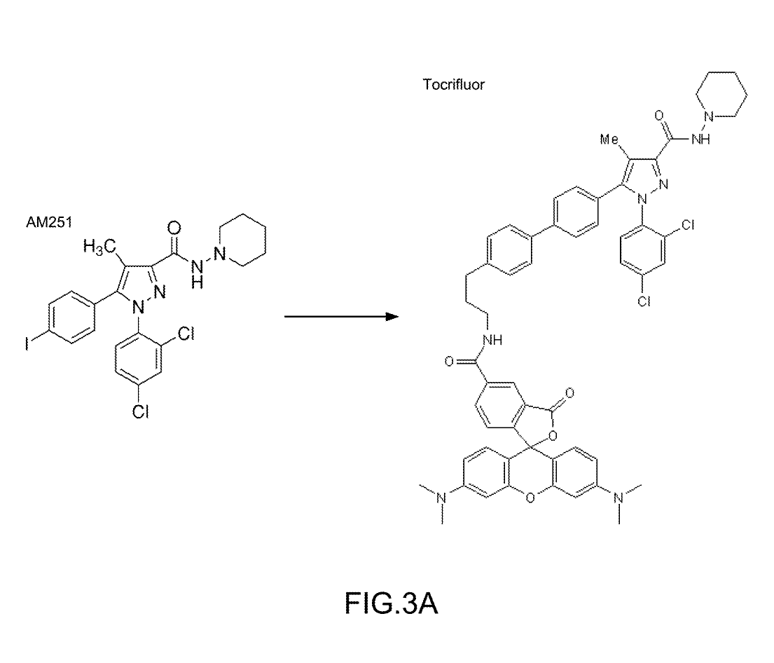

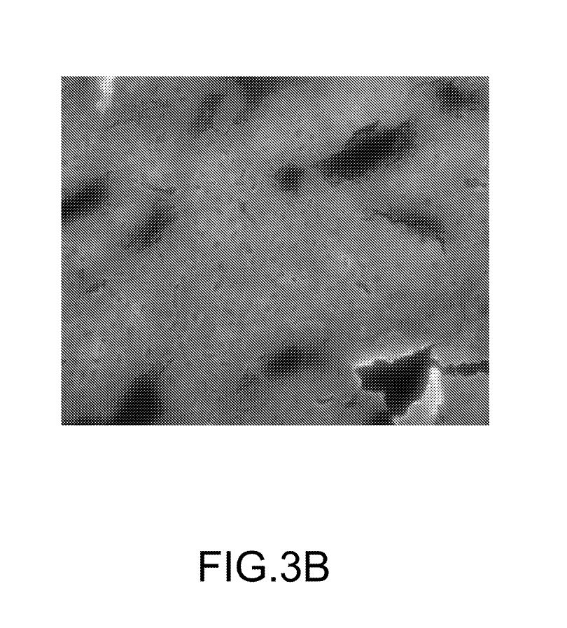

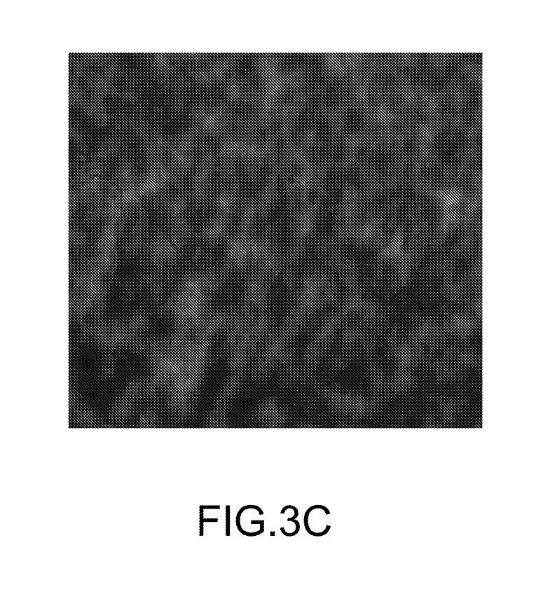

Please refer to FIG. 3A, which are a view showing a structure of Tocrifluor; and views showing images of a mouse having liver fibrosis and a normal mouse taken through fluorescence imaging. As shown in the figures, an antagonist-AM251 of CB1 receptor of liver fibrosis cell is labeled with a fluorescent substance to obtain Tocrifluor, whose chemical name is N-(Piperidin-1-yl)-5-(4-(4-(3-(5-carboxamidotetramethyl rhodaminyl)propyl))phenyl)-1-(2,4-dichlorophenyl)-4-methyl-1H-pyrazole-3-carboxamide with structure shown in FIG. 3A. The Tocrifluor is a novel fluorescent ligand in CB1 receptor, where AM251 is conjugated with 5-carboxytetramethylrhodamine (5-TAMRA) and is bonded with GPR55 at the same time. Thus, cannabinoid receptor of CB1 in liver fibrosis cell is labeled for fluorescence imaging. As a result shows in FIG. 3B and FIG. 3C, fluorescence is found at 590 nm for a 543 nm light. Thus, through fluorescence imaging, liver fibrosis level can be identified through brightness in the images according to the present disclosure.

Thus, the present disclosure diagnoses level of liver fibrosis through brightness in a liver fibrosis image with a marker of monoclonal antibody labeled with a radioactive isotope like Tc-99m, I-123, I-125, In-111 or Re-188, or a marker of antagonist labeled with a fluorescent substance for CB1 of liver fibrosis cell through a nuclear imaging like PET or SPECT. A safe, non-invasive and accurate liver fibrosis diagnosis and trace is thus done in clinic use for patients having B-type or C-type hepatitis, alcoholic hepatitis and non-alcoholic steatohepatitis to achieve early diagnosis and early treatment for preventive medicine.

To sum up, the present disclosure is a nuclear imaging method using a molecular target detection agent for liver fibrosis, where a safe, non-invasive and accurate liver fibrosis diagnosis and trace in clinic use can be done to achieve early diagnosis and early treatment for preventive medicine.

The preferred embodiment herein disclosed is not intended to unnecessarily limit the scope of the disclosure. Therefore, simple modifications or variations belonging to the equivalent of the scope of the claims and the instructions disclosed herein for a patent are all within the scope of the present disclosure.

What is claimed is:

1. A nuclear imaging method using a molecular target detection agent for liver fibrosis, comprising steps of:
(a) obtaining a monoclonal antibody or an antagonist of cannabinoid receptor of liver fibrosis cell;
(b) obtaining a marker through labeling said monoclonal antibody or said antagonist with a radioactive isotope or a fluorescent substance, respectively; and
(c) putting said marker into a body through intravenous injection and processing an in vivo imaging to said body.

2. The method according to claim 1,
wherein, in step (b), said monoclonal antibody is labeled with a radioactive nuclide selected from a group consisting of Tc-99m, I-123, I-125, In-111 and Re-188; and
wherein, in step (c), said in vivo imaging is done through a computed tomography (CT).

3. The method according to claim 1,
wherein said CT is selected from a group consisting of positron emission tomography (PET) and single photon emission computed tomography (SPECT).

4. The method according to claim 1,
wherein, in step (b), said antagonist is labeled with a fluorescent material; and
wherein, in step (c), said in vivo imaging is fluorescence imaging.
